(19) United States
(12) Patent Application Publication
(10) Pub. No.: US 2026/0174453 A1
(43) Pub. Date: Jun. 25, 2026

(54) THROMBECTOMY APPARATUS AND METHOD

(71) Applicant: Varian Medical Systems, Inc., Palo Alto, CA (US)

(72) Inventor: Francis Facchini, Hinsdale, IL (US)

(21) Appl. No.: 18/999,414

(22) Filed: Dec. 23, 2024

(51) Int. Cl. A61B 17/221; A61B 17/22; A61M 1/00

(52) U.S. Cl. CPC A61B 17/221; A61M 1/815; A61B 2017/22079; A61B 2017/2215

(57) ABSTRACT

A thrombectomy apparatus may include a non-locking syringe directly connected in fluid communication to a thrombectomy catheter.

### TECHNICAL FIELD

These teachings relate generally to treating a patient's thrombus and more particularly to conducting a thrombectomy.

### BACKGROUND

A thrombus is a clot of blood formed within a blood vessel that remains attached to its place of origin (with a plurality of such clots being referred to as thrombi). A thrombus can be the cause of a stroke. A stroke occurs when the blood supply to part of the brain is interrupted or reduced, preventing brain tissue from getting oxygen and nutrients, which can cause brain cells to die. A stroke caused by a thrombus is a significant cause of death and disability. A thrombus can occur in any other part of the body including the heart, the liver, kidney, extremities including arms and legs, and so forth.

Current mechanical thrombectomy devices include stent retrievers that are designed to capture and remove a clot by expanding within the blood vessel to entrap or otherwise engage the thrombus. When then pulled back, the device can serve to bring at least a part of the thrombus therewith.

Unfortunately, prior art approaches may be inadequate, at least in some application settings, for effectively serving as intended due to such things as limitations with respect to the application of force, thrombus positioning, and the risk of clot fragmentation, which can lead to further embolization, vessel occlusion, and resulting subsequent complications. Current thrombectomy devices also may not be sufficiently gentle on fragile blood vessels.

### BRIEF DESCRIPTION OF DRAWINGS

Various needs are at least partially met through provision of the thrombectomy apparatus and method described in the following detailed description, particularly when studied in conjunction with the drawings, wherein:

FIG. 1 comprises a block diagram as configured in accordance with the prior art; and

FIG. 2 comprises a block diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a thrombectomy apparatus can comprise a syringe connected in fluid communication to a thrombectomy catheter. By one approach, the syringe comprises a non-locking syringe. In lieu of the foregoing, or in combination therewith, the syringe can directly connect to the thrombectomy catheter. By one approach, the apparatus further comprises at least one mechanical thrombectomy component operatively coupled to the thrombectomy catheter and configured to be disposed through a patient's blood-carrying vessel to a thrombus that is to be removed from the patient.

A corresponding method can comprise disposing a thrombectomy catheter within a patient having at least one thrombus to be removed such that an inserted end of the thrombectomy catheter is located proximal to the at least one thrombus. A syringe can be connected in fluid communication to the aforementioned thrombectomy catheter. (The syringe may be non-locking and/or the syringe can be directly connected in fluid combination to the thrombectomy catheter.) A gradually increasing pressure can be applied to the syringe while monitoring a resistance to the application of pressure to the syringe and thrombectomy catheter. One or more pressure-related parameters related to the application of pressure to the syringe can be modified based on a result of the monitoring.

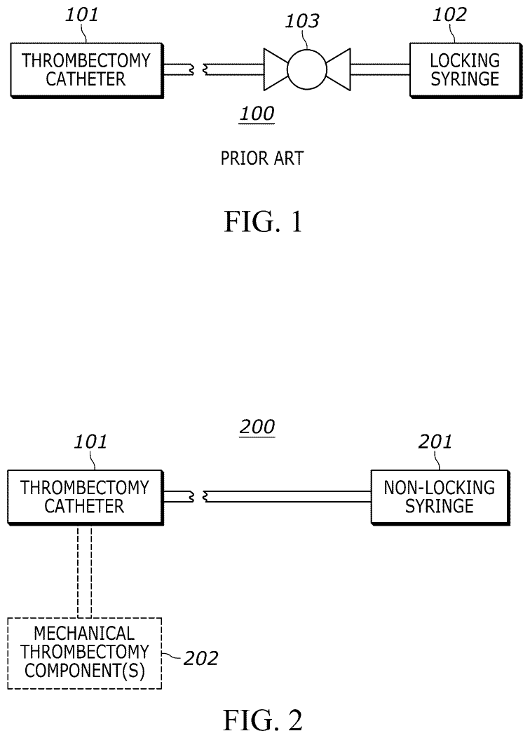

In addition to using mechanical thrombectomy components to remove thrombi, the prior art also teaches use of aspiration. FIG. 1 presents an illustrative example in these regards. In this example, a thrombectomy apparatus 100 includes a thrombectomy catheter 101 that is connected in fluid communication to a locking syringe 102 via a valve 103.

A locking syringe is a type of syringe designed with a feature that prevents the plunger from being pulled back or pushed forward beyond a predetermined point. This mechanism is intended to control the volume of fluid that can be drawn into or expelled from the syringe. The locking mechanism can take various forms, depending on the design of the syringe. For example, some locking syringes have a ratchet or notch system on the plunger that interacts with corresponding teeth or a barrier inside the barrel, which locks the plunger once it reaches a certain point. In the prior art thrombectomy process, the locking syringe is used to create a vacuum that is stored because of the combination of the locking syringe and the fluid valve which isolates the locking syringe from fluid connection with the catheter. To remove the thrombus, once the catheter is in the appropriate location proximate the blood clot, the physician opens the fluid valve which causes the entire vacuum pressure stored in the locking syringe to be applied to the catheter, aspirating at least part of the thrombus. However, the application of the entire vacuum of the locking syringe to the catheter all at once may result in a variety of complications, including the breaking up of the blood clot, the aspiration of an undesirable amount of blood or even the collapse of catheter.

The schematic symbol shown in FIG. 1 for the valve 103 suggests a ball valve. It will be understood, however, that the valve used for this purpose can comprise any of a wide variety of valve types, such as hemostasis valve or central access valves. The essential property is that the syringe 102 can be selectively blocked from fluid communication with the thrombectomy catheter 101 via such a valve.

FIG. 2 presents an illustrative example of a thrombectomy apparatus 200 that accords with the present teachings.

In this example, a syringe comprising a non-locking syringe 201 is directly connected in fluid communication to a thrombectomy catheter 101. In particular, there is no valve or other selectively-blocking mechanism between these two components. More particularly, and to be specific, “no valve” means no valve, whether fully closed, partially opened, or fully opened. Though these differences from the prior art may seem slight, their effect can be considerable.

Once the thrombectomy catheter 101 is placed in a proper position, under the control of the attending physician one or more suction actions or patterns may be applied using the non-locking syringe 201 that is in direct fluid communication to the thrombectomy catheter 101 to at least partially aspirate a targeted thrombus into the thrombectomy catheter 101.

The direct fluid connection of the non-locking syringe 201 to the thrombectomy catheter 101 provides a direct analog feedback circuit to the physician that can be sensed by the physician's hand(s). Based upon this sensory feedback, the physician can adjust one or more aspiration parameters, such as a speed by which pressure is applied or relaxed, a duration of applying or withdrawing pressure, and time intervals between applying and/or withdrawing pressure. This capability, in turn, permits the physician to tailor an aspiration operation to the characteristics of the thrombus (such as the size, location, and/or consistency of the thrombus). In some embodiments, the non-locking syringe and catheter assembly are in direct fluid connection as packaged. In other embodiments, the non-locking syringe and catheter assembly are packaged as separate components, and the physician or other attending clinician needs to fluidly connect the two elements prior to the beginning of the procedure. Although a preassembled non-locking syringe and catheter assembly may be more convenient in some cases, the unassembled non-locking syringe and catheter components allow for more flexibility, enabling the physician to mix and match different sizes of each component with one another based on the facts of the case and his or her judgment at the time of the procedure.

As a simple illustrative example, in a given procedure the physician might withdraw the plunger by hand slowly a short distance and then stop. The plunger may then be withdrawn slowly again a similar short distance following a delay of a few seconds and then again stopped. This pattern may continue a few cycles, and then the physician may pause for a longer duration of time, following which the physician pushes the plunger back into the non-locking syringe 201 a short distance. After a short pause, the physician may then again withdraw the plunger a short distance before again stopping. Variations on the foregoing can continue under the guidance of the physician until the physician is satisfied with the aspiration result.

The aforementioned direct fluid connection between the non-locking syringe 102 and the thrombectomy catheter 101 additionally enables the physician to adjust the applied suction force on the non-locking syringe 201 based on the visual clues of seeing the amount and consistency of the clot that is being collected in the non-locking syringe 201. This option is not available in systems where there is no direct fluid connection between the catheter and syringe, and where the application of the negative pressure created in a locked syringe is applied all at once and almost instantly to the catheter, and therefore not allowing any possibility of adjustment.

As noted above, that direct fluid connection between the non-locking syringe 201 and the thrombectomy catheter 101 allows the physician to selectively apply either a negative pressure or a positive pressure to the thrombectomy catheter 101 at any time during the procedure as needed. Such a capability may be precluded when using a locking syringe. In some cases, the physician may alternate between withdrawing the plunger to create suction and the pressing of the plunger to apply blowing instead of sucking and by reversing the applied pressure to the catheter. The direct fluid connection between the catheter and syringe provide additional flexibility to the physician to adjust the parameters of the thrombectomy process on the fly as he or she deems necessary to improve the results of the procedure.

So configured, these teachings will accommodate a thrombectomy method that includes disposing the thrombectomy catheter 101 within a patient having at least one thrombus to be removed from the patient such that an inserted end of the thrombectomy catheter 101 is located proximal to the at least one thrombus. The non-locking syringe 201 can be directly connected in fluid communication to the thrombectomy catheter 101. The physician can then utilize the syringe's plunger to apply, by hand, a gradually increasing (and/or decreasing) pressure to the non-locking syringe 201 while monitoring, via their hand(s), a resistance to the application of pressure to the non-locking syringe 201 and thrombectomy catheter 101. During this process, based upon what they feel and/or see, the physician can modify one or more pressure-related parameters related to the application of pressure to the non-locking syringe 201. At least a portion of the at least one thrombus is then aspirated using the application of pressure to the non-locking syringe as customized in real time during the thrombectomy by the physician based on their personal monitoring of the process.

The thrombectomy apparatus 200 can further include, if desired, one or more mechanical thrombectomy components 202. Such a mechanical thrombectomy component can be configured to be disposed through a patient's blood-carrying vessel to a thrombus that is to be removed from the patient. Such a mechanical thrombectomy component 202 may comprise, for example, a clot retriever such as an entrapment cage. The aforementioned stent retriever is one example in the foregoing regards. Such a stent retriever is configured to expand and encapsulate or capture the thrombus and to help maintain the clot structural integrity during the aspiration and removal process.

So configured, these teachings will accommodate disposing at least one mechanical thrombectomy component 202 through the thrombectomy catheter 101 and manipulating the at least one mechanical thrombectomy component 202 to remove a part of the at least one thrombus from the patient via the at least one mechanical thrombectomy component 202. This activity can be accompanied by aspiration carried out via the non-locking syringe 201 before, during, and/or after the use of the mechanical thrombectomy component 202.

So configured, the aforementioned direct fluid connection of the thrombectomy catheter 101 and non-locking syringe 201 without any intervening valve and without the need for a locking syringe provides increased and constantly available direct feedback and control to the physician to modulate the suction force. This approach can result in an improved process, reduced clot breakup, and an improved outcome for the patient.

Further aspects of these teachings are provided by the subject matter of the following clauses (where it will be understood that any of these clauses can be combined with any one of more of the other clauses as desired).

Clause 1. A thrombectomy apparatus comprising: a syringe directly connected in fluid communication to a thrombectomy catheter.

Clause 2. The thrombectomy apparatus of clause 1 wherein the syringe comprises a non-locking syringe.

Clause 3. The thrombectomy apparatus of clause 1 further comprising: at least one mechanical thrombectomy component operably coupled to the thrombectomy catheter and configured to be disposed through a patient's blood-carrying vessel to a thrombus that is to be removed from the patient.

Clause 4. The thrombectomy apparatus of clause 3 wherein the at least one mechanical thrombectomy component comprises a clot retriever.

Clause 5. The thrombectomy apparatus of clause 4 wherein the clot retriever comprises a clot entrapment cage.

Clause 6. A thrombectomy apparatus comprising: a non-locking syringe connected to a thrombectomy catheter.

Clause 7. The thrombectomy apparatus of clause 6 wherein the non-locking syringe is directly connected in fluid communication to the thrombectomy catheter.

Clause 8. The thrombectomy apparatus of clause 6 further comprising: at least one mechanical thrombectomy component operably coupled to the thrombectomy catheter and configured to be disposed through a patient's blood-carrying vessel to a thrombus that is to be removed from the patient.

Clause 9. The thrombectomy apparatus of clause 8 wherein the at least one mechanical thrombectomy component comprises a clot retriever.

Clause 10. The thrombectomy apparatus of clause 9 wherein the clot retriever comprises a clot entrapment cage.

Clause 11. A thrombectomy apparatus comprising: a non-locking syringe directly connected in fluid communication to a thrombectomy catheter.

Clause 12. The thrombectomy apparatus of clause 11 further comprising: at least one mechanical thrombectomy component operably coupled to the thrombectomy catheter and configured to be disposed through a patient's blood-carrying vessel to a thrombus that is to be removed from the patient.

Clause 13. The thrombectomy apparatus of clause 12 wherein the at least one mechanical thrombectomy component comprises a clot retriever.

Clause 14. The thrombectomy apparatus of clause 13 wherein the clot retriever comprises a clot entrapment cage.

Clause 15. A thrombectomy method comprising: disposing a thrombectomy catheter within a patient having at least one thrombus to be removed from the patient such that an inserted end of the thrombectomy catheter is located proximal to the at least one thrombus; directly connecting a syringe in fluid communication to the thrombectomy catheter; applying a gradually increasing pressure to the syringe while monitoring a resistance to the application of pressure to the syringe and thrombectomy catheter; modifying one or more pressure-related parameters related to an application of pressure to the syringe based on a result of the monitoring; aspirating at least a portion of the at least one thrombus using the application of pressure to the syringe as customized based on the monitoring.

Clause 16. The thrombectomy method of clause 15 wherein the one or more pressure-related parameters comprise at least one of a speed of applying pressure, a duration of applying pressure, and time intervals between each application of pressure.

Clause 17. The thrombectomy method of clause 15 wherein the syringe comprises a non-locking syringe.

Clause 18. The thrombectomy method of clause 15 further comprising: disposing at least one mechanical thrombectomy component through the thrombectomy catheter; manipulating the at least one mechanical thrombectomy component to remove a part of the at least one thrombus from the patient via the at least one mechanical thrombectomy component.

Clause 19. A thrombectomy method comprising: disposing a thrombectomy catheter within a patient having at least one thrombus to be removed from the patient; hand manipulating a non-locking syringe that is connected in fluid communication to the thrombectomy catheter to aspirate at least a part of the at least one thrombus.

Clause 20. The thrombectomy method of clause 19 wherein the non-locking syringe is directly connected in fluid communication to the thrombectomy catheter.

Clause 21. The thrombectomy method of clause 20 further comprising: disposing at least one mechanical thrombectomy component through the thrombectomy catheter; manipulating the at least one mechanical thrombectomy component to remove a part of the at least one thrombus from the patient via the at least one mechanical thrombectomy component.

Clause 22. A thrombectomy method comprising: disposing a thrombectomy catheter within a patient having at least one thrombus to be removed from the patient; hand manipulating a non-locking syringe that is directly connected in fluid communication to the thrombectomy catheter to aspirate at least a part of the at least one thrombus.

Clause 23. The thrombectomy method of clause 22 further comprising: disposing at least one mechanical thrombectomy component through the thrombectomy catheter; manipulating the at least one mechanical thrombectomy component to remove a part of the at least one thrombus from the patient via the at least one mechanical thrombectomy component.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A thrombectomy apparatus comprising:
a syringe directly connected in fluid communication to a thrombectomy catheter.

2. The thrombectomy apparatus of claim 1 wherein the syringe comprises a non-locking syringe.

3. The thrombectomy apparatus of claim 1 further comprising:
at least one mechanical thrombectomy component operably coupled to the thrombectomy catheter and configured to be disposed through a patient's blood-carrying vessel to a thrombus that is to be removed from the patient.

4. The thrombectomy apparatus of claim 3 wherein the at least one mechanical thrombectomy component comprises a clot retriever.

5. The thrombectomy apparatus of claim 4 wherein the clot retriever comprises a clot entrapment cage.

6. A thrombectomy apparatus comprising:
a non-locking syringe connected to a thrombectomy catheter.

7. The thrombectomy apparatus of claim 6 wherein the non-locking syringe is directly connected in fluid communication to the thrombectomy catheter.

8. The thrombectomy apparatus of claim 6 further comprising:
at least one mechanical thrombectomy component operably coupled to the thrombectomy catheter and configured to be disposed through a patient's blood-carrying vessel to a thrombus that is to be removed from the patient.

9. The thrombectomy apparatus of claim 8 wherein the at least one mechanical thrombectomy component comprises a clot retriever.

10. The thrombectomy apparatus of claim 9 wherein the clot retriever comprises a clot entrapment cage.

11. A thrombectomy apparatus comprising:
a non-locking syringe directly connected in fluid communication to a thrombectomy catheter.

12. The thrombectomy apparatus of claim 11 further comprising:
at least one mechanical thrombectomy component operably coupled to the thrombectomy catheter and configured to be disposed through a patient's blood-carrying vessel to a thrombus that is to be removed from the patient.

13. The thrombectomy apparatus of claim 12 wherein the at least one mechanical thrombectomy component comprises a clot retriever.

14. The thrombectomy apparatus of claim 13 wherein the clot retriever comprises a clot entrapment cage.

15. A thrombectomy method comprising:
disposing a thrombectomy catheter within a patient having at least one thrombus to be removed from the patient such that an inserted end of the thrombectomy catheter is located proximal to the at least one thrombus;
directly connecting a syringe in fluid communication to the thrombectomy catheter;
applying a gradually increasing pressure to the syringe while monitoring a resistance to the application of pressure to the syringe and thrombectomy catheter;
modifying one or more pressure-related parameters related to an application of pressure to the syringe based on a result of the monitoring;
aspirating at least a portion of the at least one thrombus using the application of pressure to the syringe as customized based on the monitoring.

16. The thrombectomy method of claim 15 wherein the one or more pressure-related parameters comprise at least one of a speed of applying pressure, a duration of applying pressure, and time intervals between each application of pressure.

17. The thrombectomy method of claim 15 wherein the syringe comprises a non-locking syringe.

18. The thrombectomy method of claim 15 further comprising:
disposing at least one mechanical thrombectomy component through the thrombectomy catheter;
manipulating the at least one mechanical thrombectomy component to remove a part of the at least one thrombus from the patient via the at least one mechanical thrombectomy component.

19. A thrombectomy method comprising:
disposing a thrombectomy catheter within a patient having at least one thrombus to be removed from the patient;
hand manipulating a non-locking syringe that is connected in fluid communication to the thrombectomy catheter to aspirate at least a part of the at least one thrombus.

20. The thrombectomy method of claim 19 wherein the non-locking syringe is directly connected in fluid communication to the thrombectomy catheter.

21. The thrombectomy method of claim 20 further comprising:
disposing at least one mechanical thrombectomy component through the thrombectomy catheter;
manipulating the at least one mechanical thrombectomy component to remove a part of the at least one thrombus from the patient via the at least one mechanical thrombectomy component.

22. A thrombectomy method comprising:
disposing a thrombectomy catheter within a patient having at least one thrombus to be removed from the patient;
hand manipulating a non-locking syringe that is directly connected in fluid communication to the thrombectomy catheter to aspirate at least a part of the at least one thrombus.

23. The thrombectomy method of claim 22 further comprising:
disposing at least one mechanical thrombectomy component through the thrombectomy catheter;
manipulating the at least one mechanical thrombectomy component to remove a part of the at least one thrombus from the patient via the at least one mechanical thrombectomy component.
